SEQUENCE LISTING (10 sequences)

SEQ ID NO: 1 - DNA, 21 bp, Artificial Sequence
  siRNA-GFP sense strand
gcugacccugaaguucauctt

SEQ ID NO: 2 - DNA, 21 bp, Artificial Sequence
  siRNA-GFP antisense strand
gaugaacuucagggucagctt

SEQ ID NO: 3 - DNA, 23 bp, Artificial Sequence
  siRNA-dsRed sense strand
ttaguuccaguacggcuccaauu

SEQ ID NO: 4 - DNA, 23 bp, Artificial Sequence
  siRNA-dsRed antisense strand
aauuggagccguacuggaacutt

SEQ ID NO: 5 - DNA, 21 bp, Artificial Sequence
  siRNA-AC1 sense strand
ccucacuugcaugcccucatt

SEQ ID NO: 6 - DNA, 21 bp, Artificial Sequence
  siRNA-AC1 antisense strand
ugagggcaugcaagugaggtt

SEQ ID NO: 7 - DNA, 1077 bp, Geminivirus
atgaggactcctcgttttagagttcaagccaagaatgtctttctcacatacccaaagtgttctatacccaaagaacacctgctgtcattcattcaaacactctctctcccatcaaaccctaagttcattaaaatctgtagggagctgcatcagaatggggaacctcacttgcatgccctcatccaattcgagggcaaaatcacgattacgaacaatcgtctcttcgattgtgtacacccgagctgtagcacccgtttccaccccaacattcaaggtgccaaatccagctcagatgtcaagtcctatctggataaggacggagacaccgtcgaatggggacgatttcagatcgatggacgatctgctagaggaggtcaacaatcagcgaatgatgcttacgccaaagcgcttaacagcggcagtaagtcagaggctcttaatgtcattagggaattagtcccaaaggactttgtacttcagtttcataatctaaatagtaatttagaaaggattttccaggagcccccagctccttatgtttctcccttcacatgttcttcctttgaccaagttcctgttgaacttgacgaatgggtcgctgataatgttcgggattccgctgcgcggccatggagacccaatagtattgtcatagaaggtgatagcagaacagggaagacgatatgggccagatctttaggcccacacaattacctgtgtggacacctggaccttagtccaaaggtctttaataatgatgcctggtacaacgtcattgatgacgtcgatcctcactacctaaagcactttaaggaattcatggggtcccagagggactggcagtccaacacgaaatacgggaaacccgttcaaattaaaggtggaattcccactatcttcctctgcaatccaggacctacctcgtcctataaagagttcctagacgaggaaaagcaagaagcgctaaaggcctgggcattaaagaatgcaatcttcatcaccctcacagaaccactctactcaggttccaatcaaagtcagtcacagacaattcaagaagcgagccatccggcgtag

SEQ ID NO: 8 - DNA, 1059 bp, Geminivirus
atgccgagagccggtcgttttcaaataaatgccaaaaattatttcataacctatccccgatgctccttagcaaaggaagaggccctttcccaattaaaagccatttcttacccgacgaatatcaaattcattagggtttgcagagaactacatcaggatggggtgcctcatctccatgttctcatccaattcgaaggcaagttccaatgtaccaaccccagattcttcgatctcatttccccatcccgatcaacacatttccatccgaacattcagggagctaaatcatcgtccgatgtcaaggcttacattgaaaagggaggggaatttcttgacgatggaattttccaagtcgatgccagaagtgccaggggggagggccagcatttagctcaggtatatgcagaagcgttgaatgcttcttctaaatcagaagctcttcaaattatcaaagaaaaggatccaaagtccttttttttacagttccataacatatctgctaacgcggatcgaatcttccaggctccgccacaaacttacgttagtccgttcttatcatcctcatttacgcaagtcccagaggaaatagaagtatgggtatccgaaaatatatgccgtcccgctgcgcggccatggagaccgatcagtattgttctcgaaggtgatagccgaaccgggaagacgatgtgggcccgatctctgggcccacataactatctatgtggacacctggatctgtctcccaagatatattcaaacgacgcatggtacaacgtcattgacgacgtagacccgcattatctaaagcatttcaaagaattcatgggggcccaacgagattggcaatcaaacacaaaatacggaaagcccattcaaattaaaggtgggattcccaccatcttcttatgcaatccgggccccaattcgtcctataaagaatacctagacgaggacaagaattccaatctcaagaattgggcaatcaagaatgcgctcttcatctccctcacagagccactcttctcctccaccgatcaaagccaggcacaggcaagctaa

SEQ ID NO: 9 - DNA, 717 bp, Artificial Sequence
  GFP coding sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 10 - DNA, 747 bp, Artificial Sequence
  the dsRed coding sequence
atggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgagctcaagcttcgaattctgcagtcgacggtaccgcgggcccgggatccaccggatctagataa

(19) United States
(12) Patent Application Publication
(10) Pub. No.: US 2004/0261149 A1
(43) Pub. Date: Dec. 23, 2004

(54) siRNA-mediated inhibition of gene expression in plant cells

(76) Inventors: Claude M. Fauquet, St. Louis, MO (US); Chellappan Padmanabhan, St. Louis, MO (US); Vanitharani Ramachandran, St. Louis, MO (US)

Correspondence Address: James B. Surber, Esq., Bryan Cave, LLP, Suite 3600, One Metropolitan Square, St. Louis, MO, 63102, US

(21) Appl. No.: 10/785,862

(22) Filed: Feb. 24, 2004

Related U.S. Application Data
(60) Provisional application No. 60/449,646, filed on Feb. 24, 2003.

(51) Int. Cl. C12N 15/82; C12N 15/87

(52) U.S. Cl. 800/285; 800/278

(57) ABSTRACT

The present invention relates to short interfering RNAs (siRNAs) and methods of using the same to suppress or inhibit gene expression in plant cells. In particular, the present invention relates to siRNAs and methods that can be used to suppress or inhibit viral gene expression in plant cells. Still more particularly, the present invention relates to siRNAs and methods that can be used to suppress or inhibit geminiviral gene expression in plant cells. Of course, the siRNAs of the present invention could be used to generate plants that are resistant to geminiviral infection. In addition, methods of identifying specific compositions, or siRNAs, that can be used to inhibit gene expression in plant cells generally are provided.

### CROSS-REFERENCE TO RELATED APPLICATIONS

This invention claims priority under 35 U.S.C. §119(e) from U.S. Provisional Application Ser. No. 60/449,646, filed Feb. 24, 2003, entitled, “siRNA-Mediated Inhibition of Gene Expression in Plant Cells.” The entire disclosure of U.S. Provisional Application Ser. No. 60/449,646 is incorporated herein by reference.

### BACKGROUND OF THE INVENTION

1. Field of the Invention

The invention relates to compositions and methods that can be used to suppress or inhibit gene expression in plant cells. In particular, the invention relates to compositions and methods that can be used to suppress or inhibit viral gene expression in plant cells. Still more particularly, the invention provides compositions and methods that can be used to suppress or inhibit geminiviral gene expression in plant cells. The invention further relates to compositions and methods that can be used to generate plants that are resistant to viral infection. In addition, the invention relates to methods of identifying specific compositions that can be used to inhibit gene expression in plant cells generally.

2. Description of Related Art

Posttranscriptional gene silencing (PTGS) is a sequence-specific defense mechanism that can target both cellular and viral mRNAs, and is a widely used tool for inactivating gene expression (Baulcombe, 1999; Vance and Vaucheret, 2001). PTGS is known to occur in plants (Napoli et al., 1990), while a closely related phenomenon, RNA interference (RNAi), is known to occur in a wide range of other organisms. RNA interference has been shown to occur, for example, in Caenorhabditis elegans, Neurospora crassa, Drosophila melanogaster and in mammals (Cagoni and Macino, 1997; Elbashir et al., 2001 a; Fire, 1998; Hammond et al., 2001; Sharp, 2001). In addition, transgenes and viruses have been shown to induce gene silencing in plants, and it is now believed that PTGS is a natural defense mechanism against virus accumulation (Hamilton and Baulcombe, 1999; Matzke et al., 2001).

Virus-induced gene silencing (VIGS) has been well demonstrated for a number of plant RNA viruses (Ratcliff et al., 1997; 1999; Vance and Vaucheret, 2001). The process is initiated by double-stranded RNA (dsRNA) molecules. The dsRNA molecules are possibly generated by replicative intermediates of viral RNAs or by aberrant transgene-coded RNAs, which become dsRNA by RNA-dependent RNA polymerase activity (Dalmay et al., 2000; Waterhouse et al., 2001; Xie et al., 2001). The dsRNAs are cleaved by a member of the ribonuclease III family into short interfering RNAs (siRNAs), which generally range in size from 21 to 26 nucleotides. It is believed that the siRNAs then promote RNA degradation by forming a multi-component nuclease complex RISC (RNA Induced Silencing Complex) that destroys cognate MRNA (Elbashir et al., 2001b; Tuschl et al., 1999; Zamore et al., 2000).

The siRNA is common to the various PTGS protective phenomena described to date, and is assumed to play a central protective role. In fact, it has been shown that directly introducing siRNAs of 21 nucleotides into mammalian cells can suppress expression of specific endogenous genes as well as various exogenous genes. Consequently, the use of siRNA technology is emerging as a special tool in gene therapy (Gitlin et al., 2002; Jacque et al., 2002; Lewis et al., 2002). To date, however, the use of siRNAs to effectively suppress or inhibit gene expression in plant cells has not been reported.

Geminiviruses are single-stranded DNA viruses that present a constant threat to many economically important crops (Brown, 1994; Lapidot and Friedmann, 2002). A number of pathogen-derived resistance strategies have been investigated for the control of geminiviruses, which generally involve transforming the crop to express viral coat protein, replicase (Rep), N-Rep or G5 protein (Brunetti et al., 1997; Chatterji et al., 2001; Day et al., 1991; Hong and Stanley, 1996; Kunik et al., 1994; Norris et al., 1996; Padidam et al., 1999; Sangare et al., 1999). Among these strategies, the Rep-mediated version conferred a relatively higher level of resistance when compared to the others. However, expression of the Rep protein in transgenic plants can sometimes interfere with plant phenotype.

In light of the foregoing, it would be desirable to provide methods of identifying specific siRNAs that could be used to effectively suppress or inhibit gene expression in plant cells. Moreover, it would be desirable to provide siRNAs and methods that could be used to suppress or inhibit viral gene expression in plant cells. In particular, it would be desirable to provide siRNAs and methods that could be used to suppress or inhibit geminiviral gene expression in plant cells. Of course, such compositions and methods could then be used to generate plants that are resistant to viral infection.

### BRIEF SUMMARY OF THE INVENTION

The present invention provides compositions and methods that can be used to inhibit gene expression in plant cells. In particular, the present invention provides short interfering RNAs (siRNAs) and methods that can be used to inhibit viral gene expression in plant cells. The present invention includes siRNAs and methods that can be used to suppress or inhibit geminiviral gene expression in plants cells. Of course, the siRNAs and methods of the present invention could be used in a PTGS-mediated strategy for generating virus-resistant transgenic plants. In addition, the present invention provides methods of identifying specific compositions, or siRNAs, that can be used to inhibit gene expression in plant cells generally.

In a first embodiment, siRNAs and methods of using the same to suppress or inhibit gene expression in plant cells are provided. The representative siRNAs disclosed herein are specifically targeted to the AC1 gene of African cassava mosaic virus (ACMV), which encodes the replication-associated protein (Rep) and is essential for viral DNA replication. The siRNAs are shown to interfere with viral DNA accumulation in tobacco protoplasts, and thus, are useful in suppressing or inhibiting geminiviral gene expression. Of course, the siRNAs could be incorporated into an appropriate vector, and used to generate transgenic plants that are resistant to geminiviral infection.

In a second embodiment, methods of identifying specific compositions, or siRNAs, that can be used to inhibit gene expression in plant cells generally are provided. Specifically, the methods involve designing and testing siRNAs in plant protoplasts for their ability to suppress or inhibit the expression of a target gene. These methods, for example, could be used to identify siRNAs that are capable of suppressing viral gene expression, which may be critical to viral replication. Such target sequences may comprise viral promoters, or, as shown herein, genes that encode proteins that are necessary for viral replication. The effectiveness of such methods was demonstrated by designing and testing specific siRNAs for their ability to suppress geminiviral gene expression in tobacco protoplasts—as described above. The methods were further demonstrated, in part, by designing and testing specific siRNAs for their ability to suppress transient expression of reporter genes in tobacco protoplasts.

### BRIEF DESCRIPTION OF THE DRAWINGS

FIG. 1(A) represents the genome organization of DNA A and DNA B of the African cassava mosaic virus strain Cameroon (ACMV-CM). DNA A contains six open reading frames (ORFs)—AC1, AC2, AC3, AC4, AV1 and AV2. DNA B contains BV1 and BC1 ORFs. A-stands for DNA-A; B-stands for DNA B; V for virion-sense and C for complementary-sense genes. AC1 encodes replication-associated protein (Rep). (B) and (C) are schematic representations of the AC1 gene of ACMV DNA-A and 35S-EGFP plasmid DNA, respectively. As shown, the siRNA-AC1 molecule targets (or is complementary to) nucleotides 161-181 of the AC1 gene, whereas the siRNA-GFP molecule targets (or is complementary to) nucleotides 142-162 of the green fluorescent protein (GFP) coding region.

FIG. 2. Effect of siRNA on GFP and dsRed expression in BY-2 tobacco protoplasts. dsRed is a commercially-available Reef Coral Fluorescent Protein (RCFP) (Clontech Laboratories, California, USA). The GFP and dsRed-encoding sequences were driven by the 35S cauliflower mosaic virus (CaMV35S) promoter. (A) Protoplasts transfected with only GFP plasmid DNA are represented by the bar labeled “GFP;” protoplasts transfected with GFP plasmid DNA, dsRed plasmid DNA and siRNA-GFP are represented by the bar labeled “G+R+siGFP;” and protoplasts transfected with GFP plasmid DNA, dsRed plasmid DNA and siRNA-dsRed are represented by the bar labeled “G+R+siRed.” GFP fluorescence is normalized with the auto-fluorescence of non-transfected control cells. (B) Protoplasts transfected with only dsRed plasmid DNA are represented by the bar labeled “dsRed;” protoplasts transfected with GFP plasmid DNA, dsRed plasmid DNA and siRNA-GFP are represented by the bar labeled “G+R+siGFP;” and protoplasts transfected with GFP plasmid DNA, dsRed plasmid DNA and siRNA-dsRed are represented by the bar labeled “G+R+siRed.” dsRed fluorescence is normalized with the auto-fluorescence of non-transfected control cells. Columns and bars indicate mean and standard deviation values, respectively.

FIG. 3. Effect of siRNA targeted to the AC1 gene of ACMV on ACMV and East African cassava mosaic Cameroon virus (EACMCV) DNA accumulation. Southern blots showing relative levels of viral DNA accumulation at 36 and 48 hours post-transfection in BY2 tobacco protoplasts. For co-transfection, either infectious DNA-A and DNA-B clones of ACMV or EACMCV along with (+) or without (−) siRNA-AC1 was used. 5 μg of total DNA isolated from protoplasts was loaded in each lane. The blots were probed with [∝-³²P]dCTP-labeled ACMV-specific (left panel) or EACMV-specific DNA (right panel) sequences. The hours of post-transfection is adjacent to the “h.p.t.” designation.

FIG. 4. Northern blot analysis of AC1-specific mRNA in transfected BY2 tobacco protoplasts. 10 μg of total RNA isolated from protoplasts transfected with the infectious clones of DNA-A and DNA-B of ACMV-CM along with (+) or without (−) siRNA specific for AC1 of ACMV-CM was loaded in each lane. AC1-specific [∝-³²P]dCTP-labeled DNA was used as the probe. The hours of post-transfection is adjacent to the “h.p.t.” designation. Ethidium bromide staining of ribosomal RNA shows equal loading of the samples.

### BRIEF DESCRIPTION OF THE SEQUENCES IN THE SEQUENCE LISTING

SEQ ID NO. 1: the siRNA-GFP sense strand sequence (5′ to 3′)

SEQ ID NO. 2: the siRNA-GFP antisense strand sequence (5′ to 3′)

SEQ ID NO. 3: the siRNA-dsRed sense strand sequence (5′ to 3′)

SEQ ID NO. 4: the siRNA-dsRed antisense strand sequence (5′ to 3′)

SEQ ID NO. 5: the siRNA-AC1 sense strand sequence (5′ to 3′)

SEQ ID NO. 6: the siRNA-AC1 antisense strand sequence (5′ to 3′)

SEQ ID NO. 7: the AC1 coding region of ACMV-CM (5′ to 3′)

SEQ ID NO. 8: the AC1 coding region of EACMCV (5′ to 3′)

SEQ ID NO. 9: the GFP coding sequence from EGFP-C2 expression plasmids (5′ to 3′)

SEQ ID NO. 10: the dsRed coding sequence from dsRed expression plasmids (5′ to 3′)

### DETAILED DESCRIPTION OF THE INVENTION

Geminiviruses are single-stranded DNA viruses that present a constant threat to many economically important crops. Geminiviruses have a single-stranded DNA genome that is replicated in the nuclei of infected plant cells by a rolling circle mechanism (Hanley-Bowdoin et al., 1999; Saunders et al., 1991; Stenger et al., 1991). Among the different gene products encoded by the virus, only AC1, the replication-associated protein (Rep), is essential for viral DNA replication (Fontes et al., 1994; Laufs et al., 1995; Stanley, 1995).

Double-stranded RNA (dsRNA) is remarkably effective at suppressing specific gene expression in a number of organisms, including plants. Virus-induced gene silencing (VIGS), for example, has been demonstrated for a number of plant RNA viruses (Ratcliff et al., 1997; 1999; Vance and Vaucheret, 2001). The process is initiated by double-stranded RNA (dsRNA) molecules. The dsRNA molecules are possibly generated by replicative intermediates of viral RNAs or by aberrant transgene-coded RNAs, which become dsRNA by RNA-dependent RNA polymerase activity (Dalmay et al., 2000; Waterhouse et al., 2001; Xie et al., 2001). Such dsRNA molecules have been incorporated into plants cells, and shown to be useful in suppressing or inhibiting viral gene expression (US 2002/0169298 A1, Waterhouse et al., published Nov. 14, 2002, incorporated herein by reference).

Within the plant cell, the dsRNAs are cleaved by a member of the ribonuclease III family into short interfering RNAs (siRNAs), which generally range in size from 21 to 26 nucleotides. It is believed that the siRNAs then promote RNA degradation by forming a multi-component nuclease complex RISC (RNA Induced Silencing Complex) that destroys cognate MRNA (Elbashir et al., 2001b; Tuschl et al., 1999; Zamore et al., 2000).

Recently, it has been shown that such siRNAs ranging in size from 21 to 26 nucleotides, an intermediate of the RNAi pathway, are equally effective in suppressing gene expression in animal and mammalian systems. The use of siRNAs, therefore, has become a powerful tool for down regulating gene expression and has been successfully demonstrated in a wide range of mammalian cells (Fire et al., 1998; Gitlin et al., 2002; Jacque et al., 2002; Krichevsky and Kosik, 2002; Lewis et al., 2002).

The inventors have discovered that siRNAs can be designed and used to suppress or inhibit gene expression in plant cells, which, until now, has not been reported. In particular, the inventors have developed siRNAs and methods that can be used to suppress or inhibit viral gene expression in plant cells. The use of siRNAs in this capacity eliminates the need to introduce or express the dsRNA precursor to the siRNAs, and represents a significant improvement over existing methods.

In general, when designing siRNA molecules that will be used to suppress or inhibit the expression of a particular gene, at least one strand of the two-stranded siRNA molecule must be substantially complementary to the targeted nucleic acid sequence (generally referred to as the antisense strand). The antisense strand is preferably at least 90% complementary to the targeted nucleic acid sequence, and still more preferably is 100% complementary to the targeted sequence. Of course, the second strand (or the sense strand) will be complementary to the antisense strand. In this way, the two RNA oligonucleotides can be annealed to one another to form the desired siRNA.

When designing siRNA molecules that will be used to suppress or inhibit the replication of a particular virus in plant cells, the targeted nucleic acid sequence should be critical to viral replication. Such target sequences may comprise viral promoters, or, as shown below, genes that encode proteins that are necessary for viral replication.

siRNAs can be produced chemically using methods well-known in the art. For example, the sense and antisense RNA oligonucleotides that comprise the desired siRNA molecule can be synthesized individually using well-known techniques. Alternatively, the desired RNA oligonucleotides can be purchased through various commercial vendors, such as Dharmacon Research (Lafayette, Colo., USA) or Xeragon (Madison, Wis., USA). The double-stranded siRNA molecule can then be generated by, for example, mixing the sense and antisense strands in a suitable buffer, such as 1 mM sodium citrate. The mixture can then be heated at 90° C. for one minute and then allowed to cool to room temperature.

The following non-limiting examples demonstrate the ability of siRNAs of the present invention to suppress or inhibit the expression of various genes in plant cells. Specifically, siRNAs were delivered directly into Nicotiana tabacum protoplasts along with sequences encoding the well-known green fluorescent protein (GFP) using standard electroporation techniques. The siRNAs were designed to target the GFP coding sequence (the EGFP-C2). Additionally, siRNAs were delivered directly into N. tabacum protoplasts along with sequences encoding the well-known dsRed protein using standard electroporation techniques. The siRNAs were designed to target the dsRed coding sequence. Furthermore, siRNAs were delivered directly into N. tabacum protoplasts along with the DNA genome of the African cassava mosaic virus strain Cameroon (ACMV-CM) using standard electroporation techniques. The siRNAs were designed to target the AC1 gene of ACMV-CM, which corresponds to the only viral encoded protein essential for geminiviral replication.

The effect of the siRNAs on GFP, dsRed and AC1 gene expression was quantitatively evaluated. In each case, the siRNA delivered to the plant cells resulted in a sequence specific, down-regulation of the targeted gene.

### EXAMPLES

### Example 1

### Constructs

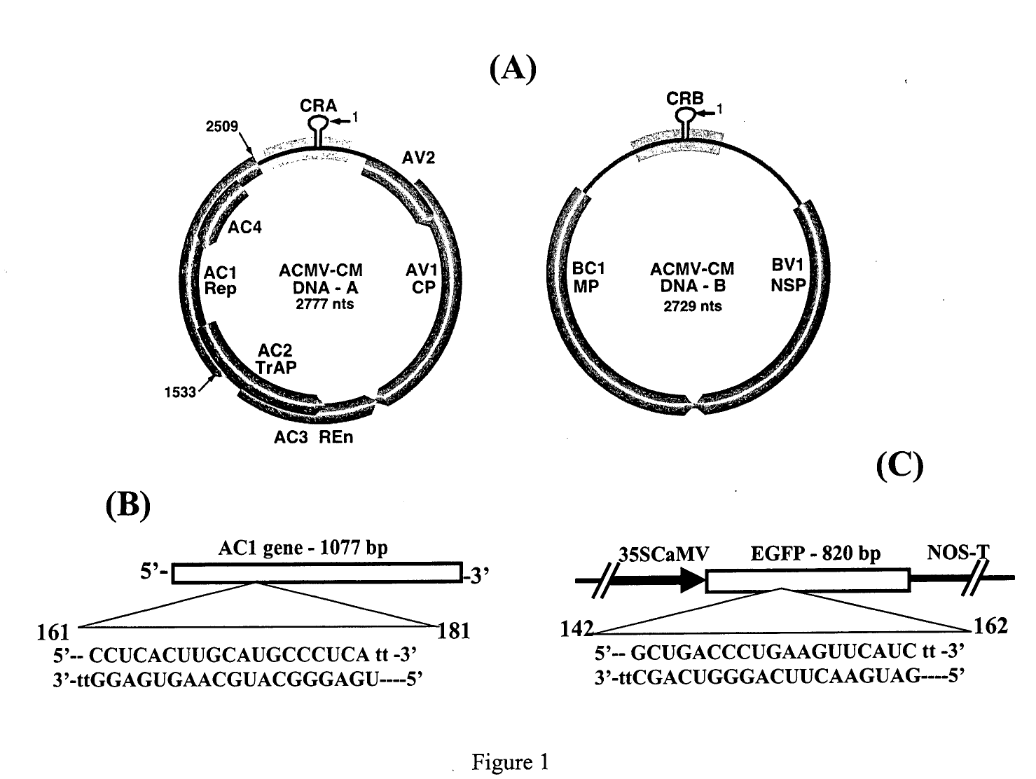

Constructions of infectious clones of DNA-A and DNA-B of African cassava mosaic virus strain Cameroon (ACMV-CM) (FIG. 1A) and East African cassava mosaic Cameroon virus (EACMCV) were prepared in accordance with methods well-known in the art (Fondong et al., 2000). GFP and dsRed-encoding sequences were derived from pEGFP-C2 and dsRed2, respectively (Clontech Laboratories, California, USA). Both GFP and dsRed are well-known, commercially available fluorescent markers. The plasmids pEGFP-C2 and dsRed2 were first transformed into a methylation minus E. coli strain JM110 using methods well-known in the art. The GFP and dsRed coding sequences were then excised as NheI and BclI fragments and ligated (Roche Diagnostics) downstream of a 35SCaMV promoter at AvrII site and BamHI site and upstream of the NOS terminator in a pUC18 based vector (FIG. 1C). This vector was then transformed into TOP10 E. coli (Invitrogen, USA).

### Example 2

### siRNA Preparation and Protoplast Transfection

Protoplasts are often employed for rapid and quantitative analysis of transgene expression (Lapidot and Friedmann, 2002; Reichel et al., 1996). In addition, gene silencing has been shown in plant protoplasts using dsRNA expression plasmids (Akashi et al., 2001). Furthermore, protoplasts are often used for studying the molecular process of viral replication in plant cells (Chatterji et al., 2001). For these reasons, representative siRNAs of the present invention were introduced into N. tabacum protoplasts using standard electroporation techniques.

In this non-limiting example, the siRNAs were targeted to the coding regions of GFP, dsRed or the AC1 gene of ACMV. The siRNAs were designed with 5′ phosphate, 3′ hydroxyl, and two base overhangs at either the 3′- or 5′-end of each strand. As described above, siRNAs can be produced chemically using methods well-known in the art. In this example, the siRNAs were chemically synthesized by Xeragon (Madison, Wis., USA). The following sequence was used to specifically target GFP expression: the siRNA-GFP sense strand consisted of 5′-GCUGACCCUGAAGUUCAUCtt-3′ (SEQ ID NO. 1) and the antisense strand consisted of 5′-GAUGAACUUCAGGGUCAGCtt-3′ (SEQ ID NO. 2). The anstisense strand was complementary to coordinates 124-144 of the GFP coding region described herein (SEQ ID NO. 9) (Note that SEQ ID NO. 9 does not include the leader sequence that is incorporated in the diagram shown in FIG. 1C, which explains the difference in the indicated nucleotide coordinates to which the siRNA-GFP antisense strand was complementary). The following sequence was used to specifically target dsRed expression: the siRNA-dsRed sense strand consisted of 5′-ttAGUUCCAGUACGGCUCCAAUU-3′ (SEQ ID NO. 3) and the antisense strand consisted of 5′-AAUUGGAGCCGUACUGGAACUtt-3′ (SEQ ID NO. 4).

The following sequence was used to specifically target the ACMV AC1 coding region: the siRNA-AC1 sense strand consisted of 5′-CCUCACUUGCAUGCCCUCAtt-3′ (SEQ ID NO. 5) and the antisense strand consisted of 5′-UGAGGGCAUGCAAGUGAGGtt-3′ (SEQ ID NO. 6) (See also FIGS. 1B, 1C). The antisense strand was complementary to coordinates 161-181 of the ACMV AC1 coding region described herein (SEQ ID NO. 7). For SEQ ID NO. 1-6, the “tt” portion at the 5′ or 3′-end of each strand constitutes the two base overhang mentioned above. The respective sense and antisense strands were incubated at 90° C. for one minute and at room temperature for one hour. This allowed the respective sense and antisense strands to anneal, and to form the desired double-stranded siRNA molecules.

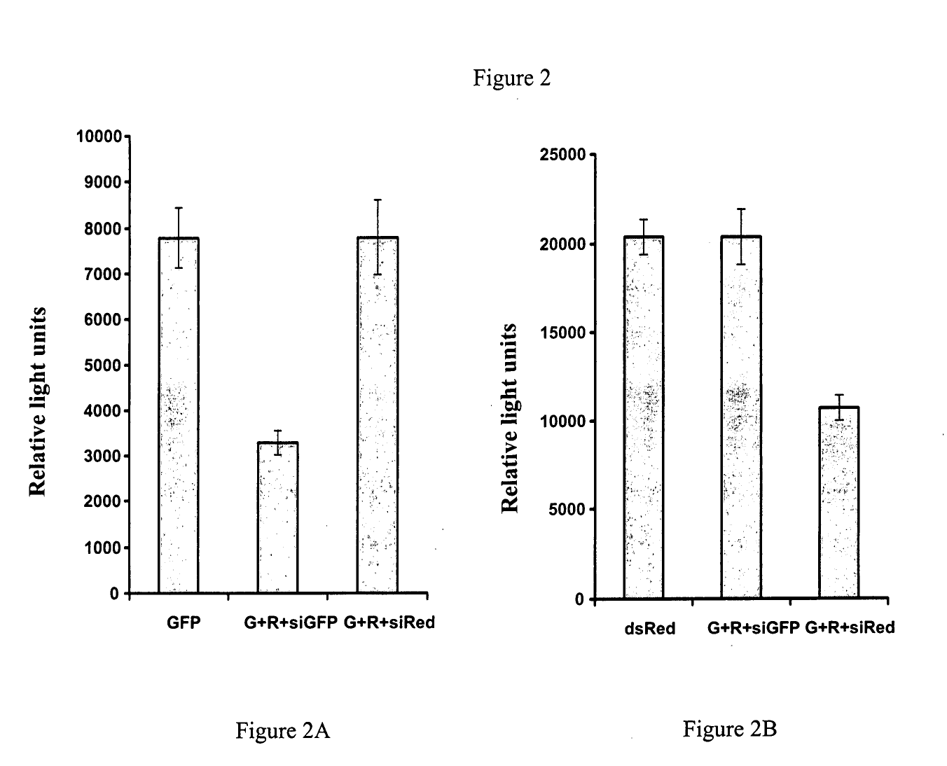

Protoplasts were isolated from three-day-old tobacco BY-2 suspension cells derived from N. tabacum (Nagata et al., 1992). The protoplasts were transfected with (i) 5 μg of GFP plasmid DNA (represented by the bar labeled “GFP” in FIG. 2A), (ii) 5 μg of GFP plasmid DNA, 5 μg of dsRed plasmid DNA and 3 μg of siRNA-GFP (represented by the bar labeled “G+R+siGFP” in FIG. 2A), (iii) 5 μg of GFP plasmid DNA, 5 μg of dsRed plasmid DNA and 3 μg of siRNA-dsRed (represented by the bar labeled “G+R+siRed” in FIG. 2A), (iv) 5 μg of dsRed plasmid DNA (represented by the bar labeled “dsRed” in FIG. 2B), (v) 5 μg of GFP plasmid DNA, 5 μg of dsRed plasmid DNA and 3 μg of siRNA-GFP (represented by the bar labeled “G+R+siGFP” in FIG. 2B), or (vi) 5 μg of GFP plasmid DNA, 5 μg of dsRed plasmid DNA and 3 μg of siRNA-dsRed (represented by the bar labeled “G+R+siRed” in FIG. 2B) (Akashi et al., 2001; Chatterji et al., 2001; Watanabe et al., 1987). In addition, protoplasts were transfected with either 4 μg each of DNA A and DNA B components of ACMV or 4 μg each of DNA A and DNA B components of EACMCV—each with or without 3 μg of siRNA-AC1.

The protoplasts (1.5 million) were electroporated with the foregoing combinations along with 20 μg of sheared herring sperm DNA at 300V, 125 microfarads using an electroporater (Bio-Rad, Hercules, Calif., USA). Protoplasts were harvested at different time intervals after electroporation for DNA, RNA and microscopic analysis.

### Example 3

### siRNA-mediated Inhibition of GFP and dsRed Gene Expression

GFP and dsRed fluorescence in protoplasts were measured using a fluorometer (FLUOstarOPTIMA, BMG Labtechnologies, Durham, N.C., USA). As described in the previous Example, protoplasts were transfected with p35S-EGFP and/or p35S-dsRed plasmid DNA along with or without siRNA-GFP or siRNA-dsRed. After 24 hours post-transfection, protoplasts were collected by centrifugation at 1000 rpm for·10 minutes at 28° C. Cells were re-suspended in 150 μl of extraction buffer (50 mM NaHPO₄ buffer, pH 7.0, 10 mM EDTA, 0.1% Triton X-100, 0.1% Sodium Lauryl Sarcosine and 1 mM Dithiothreitol (DTT)) and sonicated for a total period of 14 seconds.

Samples were kept on ice at all times, except during the sonication. The samples were then subjected to centrifugation at 12,000 rpm for 10 minutes at 4° C. The supernatant fluid was transferred to a new tube and left on ice. To 50 μl of extraction buffer in each test well of a 96-well plate, 40 μl of cell extract was added, mixed and incubated in the dark at room temperature for 30 minutes. GFP and dsRed fluorescence were measured using an excitation at 485 nm and emission at 520 nm for GFP and 544 nm and 580 nm excitation and emission, respectively, for dsRed.

In protoplasts co-transfected with GFP plasmid DNA and the corresponding siRNA cognate to GFP (siRNA-GFP), a 58% reduction of GFP fluorescence was observed, whereas protoplasts co-transfected with GFP plasmid DNA and siRNA-dsRed did not exhibit a significant reduction of GFP fluorescence (FIG. 2A). In protoplasts co-transfected with dsRed plasmid DNA and siRNA-dsRed, a 47% reduction of dsRed fluorescence was observed, whereas protoplasts co-transfected with dsRed plasmid DNA and siRNA-GFP did not exhibit a significant reduction of dsRed fluorescence. The foregoing indicates that siRNA-mediated inhibition of gene expression in plant cells is sequence specific (FIG. 2B).

Fluorescence microscopy with laser scanning was also used to capture images of the electroporated protoplasts. The effect of siRNA on GFP expression was monitored at 24 hours post-transfection. To that end, protoplasts were first harvested by centrifugation at 1000 rpm for 10 minutes and re-suspended in 500 μl of culture medium. Next, 30 μl of the protoplast suspension were transferred onto a glass slide and monitored under a microscope. Images were recorded with equal exposure time under non-saturated conditions for 141 randomly chosen GFP expressing protoplasts. Gene expression was quantified in both control protoplasts (which were transfected with GFP plasmid DNA, but not siRNA-GFP) and protoplasts transfected with GFP plasmid DNA and siRNA-GFP (Krichevsky and Kosik, 2002; Reichel et al., 1996). Quantification of pixel intensity was performed using ImageJ software and normalized to a background.

For comparison, at least 141 randomly chosen transfected control and targeted (protoplasts transfected with GFP-encoding sequences and siRNA-GFP) cells were quantified. The results revealed that GFP expression was higher in control cells compared to targeted cells, which further demonstrates that the siRNAs interfere with GFP gene expression. Microscopic results correlated well with the fluorometric biochemical assay.

### Example 4

### siRNA-mediated inhibition of geminiviral gene expression

The following non-limiting example demonstrates the ability of the siRNAs to reduce virus accumulation in protoplasts. Specifically, siRNAs targeted to the coding region (AC1) of the replication-associated protein (Rep) of ACMV-CM strain was designed as described above. As described in Example 2, protoplasts were co-transfected with the following combinations: (i) infectious clones of ACMV DNA-A and DNA-B along with or without siRNA cognate for the AC1 gene of ACMV (siRNA-AC1) or (ii) infectious clones of EACMCV DNA-A and DNA-B along with or without siRNA-AC1. Total DNA was isolated from protoplasts at 36 and 48 hours post-transfection and viral DNA accumulation was determined using Southern blot hybridization.

Southern blotting was performed as described in Sambrook and Russell (2001). Five μg of total DNA isolated from the electroporated protoplasts described above was electrophoresed on 1% agarose gel in 1×TBE without ethidium bromide and transferred to HybondN+ membrane (Amersham plc, Buckinghamshire, UK). To construct an ACMV-specific probe, a 794 bp EcoRI fragment (coordinates 1789-2583 nts) of ACMV-Ug1 DNA-A was used. To construct an EACMCV-specific probe, a 944 bp EcoRI fragment (coordinates 1821-2765 nts) of EACMV-Ug2 DNA-A was used (Pita et al., 2001). The DNA fragments were labeled using [∝-³²P]dCTP and a random primer labeling kit (Prime II kit from Stratagene, La Jolla, Calif., USA). Hybridization was carried out at 65° C. Post-hybridization washes were performed sequentially with 2×SSC, 0.5×SSC and 0.2×SSC along with 0.1% SDS, each 30 minutes at 65° C. Blots were scanned using an Amersham Storm PhosphorImager and quantified using IQMacV1.2 software (Amersham plc, Buckinghamshire, UK).

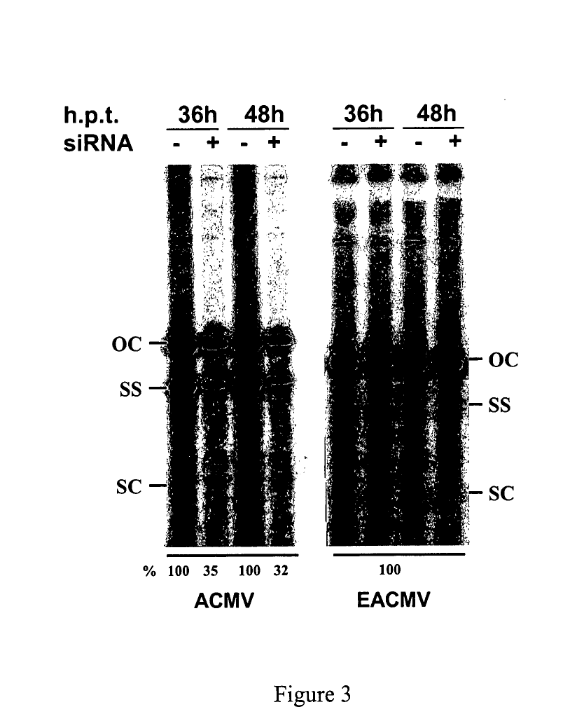

A 65% and 68% reduction in viral DNA accumulation was observed when compared to the control (protoplasts transfected with viral DNA, but not cognate siRNA) at 36 and 48 hours post-transfection, respectively (FIG. 3, left panel). This shows that siRNAs can inhibit ACMV DNA accumulation in protoplasts. The siRNAs did not interfere, however, with EACMCV DNA accumulation (FIG. 3, right panel), which is another Geminivirus species that infects cassava. ACMV siRNA-AC1 differs from its EACMCV-AC1 counterpart by only seven nucleotides (the EACMCV-AC1 coding region is set forth in SEQ ID NO. 8). This clearly demonstrates that the siRNA was highly specific for the intended ACMV target.

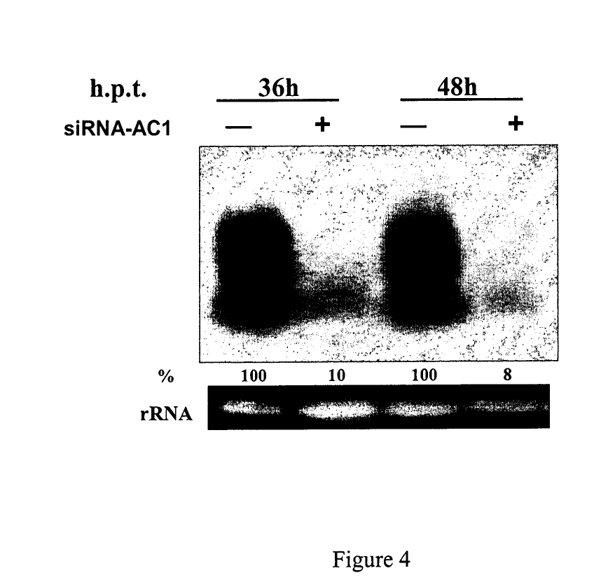

The effect of siRNA on the targeted MRNA stability in protoplasts was also evaluated by northern blot hybridization. First, 10 μg of total RNA was isolated from protoplasts transfected with infectious clones of DNA-A and DNA-B of ACMV along with or without siRNA-AC1 using an RNA isolation kit (Qiagen Inc., Valencia, Calif., USA). The RNA was electrophoresed using a 1% formaldehyde agarose gel and transferred to HybondN+ membrane. The northern blot hybridization was conducted at 42° C. using the AC1 gene specific probe described above. The northern blot hybridizations were washed and scanned using the methods described above for Southern blotting. A 90% and 92% reduction in AC1 mRNA level compared to control was observed at 36 hours and 48 hours post-transfection, respectively (FIG. 4). Of course, this suggests that siRNA-AC1 specifically degraded the complementary-sense poly-cistronic transcript of ACMV DNA-A, which encompassed the AC1 messenger.

Thus, it is clear that the siRNAs and methods disclosed herein can be used to inhibit gene expression in plant cells. In particular, the foregoing demonstrates that the siRNAs and methods disclosed herein can be used to inhibit viral gene expression in plant cells, and specifically geminiviral gene expression. That is, by interfering with the mRNA for the Rep protein, the siRNA-AC1 molecule was shown to suppress or inhibit the replication process of the geminivirus. The siRNAs and methods disclosed herein, of course, could be used to develop siRNA-based vectors, which could then be used to generate plants that are resistant to geminiviral infection. More importantly, however, the foregoing demonstrates that the siRNAs and methods of the present invention could be adapted to suppress or inhibit the expression of any endogenous or exogenous gene in plant cells.

Furthermore, the experimental systems disclosed herein could be used for rapid evaluation and study of siRNAs and their ability to down regulate specific gene expression. That is, the identity of siRNAs which could be used to suppress gene expression in plant cells is never readily apparent. In fact, the inventors have found that many different siRNAs must be tested to identify an optimal siRNA that is capable of suppressing the expression of a target gene. The use of the experimental systems described above, and particularly the use of plant protoplasts in this context, provides an efficient means of defining such optimal siRNAs. Of course, such systems would also be a valuable tool for investigating gene regulation in plants.

In addition, the experimental systems described above could be used to define multiple siRNAs that inhibit the expression of several genes that comprise, for example, a virus genome. The multiple siRNAs could then be combined and incorporated into an appropriate vector. The vector, of course, could be used to generate plants that are capable of suppressing or inhibiting the expression of the several corresponding target genes. The combined siRNAs would, in this example, provide plants with an enhanced ability to combat viral infection.

Various modifications and alterations of this invention will become apparent to those skilled in the art without departing from the scope and principles of this invention, and it should be understood that this invention is not to be unduly limited to the illustrative embodiments set forth hereinabove. All nucleotide sequences identified in this text by their SEQ ID NO are hereby incorporated by reference.

### REFERENCES

Akashi, H., Miyagishi, M. & Taira, K. (2001) Antisense & Nuc. Acid Drug Dev. 11, 359-367.

Ananthalakshmi, R., Pruss, G. J., Ge. X., Marathe, R., Mallory, A. C., Smith, T. H. & Vance, V. B. (1998) Proc. Natl. Acad. Sci. USA 95, 13079-13084.

Baulcombe, D. C. (1999) Curr. Biol. 9, R599-R601.

Brown, J. K. (1994) FAO Plant Prot. Bull. 42, 3-32.

Brunetti, A., Tavazza, M., Noris, E., Tavazza, R., Caciagli, P., Ancora, G., Crespi, S. & Accotto, G. P. (1997) MPMI 10, 571-579.

Cagoni, C & Macino, G. (1997) Proc. Natl. Acad. Sci. 94, 10233-10238.

Chatterji, A., Beachy, R. N. & Fauquet, C. M. (2001) J. Biol. Chem. 276, 25631-25638.

Dalmay, T., Hamiltom, A., Rudd, S., Angell, S. & Baulcombe, D. C. (2000) Cell 101, 543-553.

Day, A. G., Bejarano, E. R., Buck, K. W., Burrell. M. & Lichtenstein, C. P. (1991) Proc. Natl. Acad. Sci. USA 88, 6721-6725.

Elbashir, S. M., Lendeckel, W. & Tuschl, T. (2001a) Genes Dev. 15, 188-200.

Elbashir, S, M., Martinez, J., Patkaniowska, A., Lendeckel, W. & Tuschl. T. (2001b) EMBO J 20, 6877-6888.

English, J., Mueller, E. & Baulcombe, D. C. (1996) The Plant Cell 8, 179-188.

Fauquet, C. M., & Stanley, J. (2003) Ann. Appl. Biol. (In press)

Fire, A., Xu, S., Montgomery, M. K., Kostas, S. A., Driver, S. E. & Mellow, C. C. (1998) Nature 391, 806-811.

Fondong, V. N., Pita, J. S., Rey, M. E. C., Kochko A. de., Beachy, R. N & Fauquet, C. M. (2000) J. Gen. Virol. 81, 287-297.

Fontes, E. P. B., Gladfelter, H. J., Schaffer, R. L., Petty, I. T. D & Hanley-Bowdoin, L. (1994) Plant Cell 6, 405-416.

Gitlin, L., Karelsky, S. & Andino, R. (2002) Nature 418, 430-434.

Hamilton, A. J. & Baulcombe, D. C. (1999) Science 286, 950-952.

Hammond, S. M., Caudy, A. A. & Hannon, G. J. (2001) Nat. Rev. Genet. 2, 110-119.

Hanley-Bowdoin, L., Settlage, S. B., Orozco, B. M., Nagar, S. & Robertion, D. (1999) Crit. Rev. Plant Sci. 18, 71-106.

Hong, Y. & Stanley, J. (1996) MPMI 9, 219-225.

Hutvagner, G. & Zamor, P. D. (2002) Curr. Opin. Genet. Dev. 12, 225-232.

Kjemtrup, S., Sampson, K. S., Peele, C. G., Nguyen, L. V., Conkling, M. A., Thompson, W. F & Robertson, D. (1998) The Plant J. 14, 91-100.

Krichevskey, A. M. & Kosik, K. S. (2002) Proc. Natl. Acad. Sci. USA 99, 11926-11929

Kunik, T., Salomon, R., Zamir, D., Novet, N., Zeidan, M., Michelson, I., Gafni, Y. & Czosneck, H. (1994) Biotechnology 12, 500-504.

Lapidot, M & Friedmann, M (2002) Ann. Appl. Biol. 140, 109-127.

Laufs, J., Traut, W., Heyraud, F., Matzeit, V., Rogers, S. G., Schell, J. & Gronenborn, B. (1995) Proc. Natl. Acd. Sci. USA 92, 3879-3883.

Lewis, D. V., Hagstrom, J. E., Loomis, A. G., Wolf, J. A. & Herweijer, H. (2002) Nature Genetics 32, 107-108.

Mallory, A. C., Reinhart, B. J., Bartel, D. P., Vance, V. B. & Bowman L. H. (2002) Proc. Natl. Acad. Sci. USA 99, 15228-15233.

Matzke, M. A., Matzke, A. J., Pruss, G. J. & Vance, V. B. (2001) Curr. Opin. Genet. Dev. 11, 221-227.

Nagata, T., Nemoto, Y. & Haszawa, S. (1992) Int Rev Cytol. 132, 1-30

Napoli, C., Lemieux, C. & Jourgensen, R. (1990) Plant Cell 2, 279-289.

Noris, E., Accotto, G. P., Tavazza, R., Brunetti, A., Crespi, S. & Tavazza, M. (1996) Virology 224, 130-138.

Padidam, M., Beachy, R. N & Fauquet, C. M. (1999) J. Virol. 73, 1609-1616.

Peele, C., Jordan, C. V., Muangsan, N., Tunage, M., Egelkrout, Erin., Eagle, P., Hanley-Bowdoin, L. & Robertion, D. (2001) The Plant J. 27, 357-366.

Pita, J. S., Fondong, V. N., Sangare, A., Otim-Nape, G. W., Ogwal, S. & Fauquet, C. M. (2001) J. Gen. Virol. 82, 655-665.

Ratcliff, F., Harrison, B. D. & Baulcombe, D. C. (1997) Science 276, 1558-1560.

Ratcliff, F. G., MacFarlane, S. A. & Baulcombe, D. C. (1999) The Plant Cell 11, 1207-1215.

Reichel, C., Mathur, J., Eckes, P., Langenkemper, K., Koncz, C., Schell, J., Reiss, B & Maas, C (1996) Proc. Natl. Acad. Sci. USA 93: 5888-5893.

Sambrook, J., Fritsch, E. F. & Maniatis. T. (2000) Molecular cloning: a laboratory manual, 2^{nd} Ed. Cold Spring Harbor Laboratory Press, Cold Spring Harbor, N. Y.

Sangare, A., Deng. D., Fauquet, C. M. & Beachy, R. N. (1999) Mol. Biol. Re. 5,95-102.

Saunders, K., Lucy, A. & Stanley, J. (1991) Nuc. Acids Res. 19, 2325-2330.

Sharp, P. A. (2001) Genes Dev. 15, 485-490.

Stanley, J. (1995) Virology 206, 707-712.

Stenger, D. C., Revington, G. N., Stevenson, M. C. & Bisaro, D. M. (1991) Proc. Natl. Acad. Sci. USA 88, 8029-8033.

Turnage, M. A., Muangsan, N., Peele, G. C. & Robertson, D. (2002) The Plant J. 30, 107-114.

Tuschl, T., Zamore, P. D., Lehmann., R., Bartel, D. P. & Sharp, P. A. (1999) Genes Dev. 13, 3191-3197.

Vance, V. & Vaucheret, H. (2001) Science 292, 2277-2280.

Voinnet, 0. (2001) Trends Genet. 17, 449-459.

Voinnet, 0. & Baulcombe, D. C. (1997) Nature 387, 553.

Voinnet, O., Pinto, Y. M . & Baulcombe, D. C. (1999) Proc. Natl. Acad. Sci. 96, 14147-14152.

Watanabe, Y., Meshi, T. & Okada, Y. (1987) FEMS Lett. 219, 65-69.

Waterhouse, P. M., Wang, M. B. & Lough. T. (2001) Nature 411, 834-842.

Zamore, P. D., Tuschl, T., Sharp, P. A & Bartel, D. P. (2000) Cell 101, 25-33.

We claim:

1. A method to suppress the expression of a target gene in a plant cell comprising introducing a short interfering RNA (siRNA) into the plant cell, wherein the siRNA is a double-stranded molecule with a first strand consisting essentially of a nucleic acid sequence which is substantially complementary to a nucleic acid sequence of the target gene and a second strand consisting essentially of a nucleic acid sequence which is substantially complementary to said first strand, wherein said siRNA is capable of suppressing the expression of the target gene.
